(19) United States
(12) Patent Application Publication
(10) Pub. No.: US 2002/0055463 A1
(43) Pub. Date: May 9, 2002

(54) Pseudopeptide compounds having an inhibiting activity with respect to paths activated by proteins with active tyrosine kinase activity and pharmaceutical compositions containing same

(76) Inventors: Christiane Garbay, Paris (FR); Wang-Qing Liu, Paris (FR); Michel Vidal, Montrouge (FR); Bernard P. Roques, Paris (FR)

Correspondence Address: LARSON & TAYLOR, PLC, 1199 NORTH FAIRFAX STREET, SUITE 900, ALEXANDRIA, VA, 22314, US

(21) Appl. No.: 09/977,349

(22) Filed: Oct. 16, 2001

Related U.S. Application Data
(63) Continuation-in-part of application No. 09/869,191, filed on Sep. 25, 2001.
Application No. 09/869,191 is a 371 of international application No. PCT/FR99/03289, filed on Dec. 24, 1999.

(30) Foreign Application Priority Data
Dec. 24, 1998 (FR) 98 16459

(51) Int. Cl. A61K 38/06; C07K 5/08

(52) U.S. Cl. 514/7; 514/18; 530/331

(57) ABSTRACT

The invention concerns a compound corresponding to general formula (1) wherein, in particular, R₂ represents a phenylmethyl or naphthylmethyl or cyclohexylmethyl radical, 2- and 3-pryidinylmethyl, substituted on the cycle in meta or para position by an acid group or an alkyl radical of the type (CH₂)ₙ (with n=3 or 4) substituted in terminal position by an acid group; and R₃ represents a hydrogen group or a linear or branched C₁-C₄ alkyl or alkylcycloalkyl with a C₃-C₆ cycloalkyl. The invention also concerns the use of a compound of general formula (I) for preparing a pharmaceutical composition for treating diseases related to proliferative processes, cancers and/or metastases.

The present invention relates to new pseudopeptide compounds having an inhibiting effect on pathways activated by proteins with a tyrosine kinase activity and pharmaceutical compositions containing them.

Prospecting for new therapeutic agents to be used against cancers and/or diseases associated with proliferative disorders is currently one of the most important research aspects of the pharmaceutical industry. A new approach for identifying new compounds sets out to inhibit receptors of growth factors having a tyrosine kinase activity which are involved in the signal transmission process.

It is known that cell signalling, transmitted by external factors, of the growth factor, hormone and neurotransmitter variety, to transmembrane receptors, notably having a tyrosine kinase TK activity is relayed by recognition signals which involve cytoplasmic transmitters such as proteins, nucleotides and ions, before reaching the nucleus and activating the transcription factors. Of these cytoplasmic transmitters, proteins with a tyrosine kinase activity, which may or may not be receptors, play an essential part by phosphorylating cell proteins on tyrosine residues and activating cascades of kinase proteins leading to cell division and/or differentiation.

The tyrosine kinase proteins transmit their message by series of interactions which involve, for example, tyrosine residues of their C-terminal end which are capable, together with a number of amino acids which surround them, of recognising their targets. Two types of recognition have been reported, depending on whether the phosphotyrosine region recognised involves the amino acids which precede or succeed the phosphotyrosine residue (pTyr). These are, respectively, the PTB domains (phosphotyrosine binding domain) and the SH2 domains (Src homology²). Numerous proteins contain PTB and/or SH2 domains and are involved in cell signalling and neoplasic transformation. The protein Grb2 (Growth factor receptor binding protein) is one of them.

The adaptor protein Grb2 plays a direct part in the transmission of the mitogenic signal between the receptors having a tyrosine kinase activity and the Ras signalling pathway. Grb2 is complexed by its SH3 domains with Sos (Son of Sevenless), the Ras nucleotide exchange factor, and by its SH2 domain with receptors having a TK activity, directly or via other adaptors such as Shc (SH2 domain containing adaptor protein). When the receptor is activated, Grb2/Sos is recruited to the membrane and is then capable of activating Ras (Review in Chardin et al. FEBS Letters, 1995, 369, 47-51).

The importance of Grb2 in regulating the Ras-dependent signalling pathway has been demonstrated in the case of tumours which overexpress the EGF receptor (Epidermal growth factor) (breast tumours—Sastry et al., Int. J. Cancer. 1997, 70, 208-213) or its oncogenic analogue HER2 (bladder tumours; Janes et al., Oncogene, 1994, 9, 3601) or the oncogene Bcr/Abl (lymphoblastic myeloid leukaemias. J. Exp. Med., 1994, 179, 167-175). Accordingly, amplification of the gene of Grb2 was shown in certain breast cancers (Proc. Natl. Acad. Sci., 1994, 91, 2156-2160) and overexpression of the protein in certain tumoral lines (MCF7, MCA-MB361, Oncogene, 1994, 9, 2723).

Contrary to most of the SH2 domains in which the specificity is a function of the nature of the amino acid in the +3 position relative to phosphotyrosine, the specificity of recognition of the SH2 domain of Grb2 with the tyrosine kinase proteins is imposed by the amino acid in the +2 position, for which an asparagine Asn residue is particularly favourable (Cussac et al. EMBO J. 1994, 13, 4011-21). Moreover, for sterical reason, the peptide adopts a (β-turn) configuration involving the CO of pTyr and the NH of the amino acid +3 and has two anchoring sites at the amino acids phosphotyrosine and asparagine (Rahuel et al., Nature Struct. Biol., 1996, 3, 586-589).

Consequently, in the light of all this information, numerous efforts have been made in an attempt to counteract the interaction between the activated receptors containing the phosphotyrosine residue (pTyr) and the Grb2 protein at the SH2 domain of the latter. Thus, molecules capable of binding to SH2 domain of Grb2 were obtained using chemical libraries (Müllere et al., J. Mol. Biol., 1996, 28, 16500-16505), or phages (Gram, Eur. J. Biochem., 1997, 246, 633-637), or by modification of the original peptides, based on the structural data of the complexes mentioned hereinbefore.

Moreover, pseudopeptide molecules having strong affinities for the SH2 domains were obtained, particularly with the Src proteins (Pacofsky et al., J. Med. Chem., 1998, 41,1894-1908).

Within the scope of the present invention, a family of compounds is proposed which have a very strong affinity for Grb2 thanks in particular to the introduction of a negatively charged aromatic residue and blockage of the configuration of these molecules by loading the α carbon with one of the residues involved in the interaction.

More precisely, the compounds proposd within the scope of the present invention correspond to the general formula I

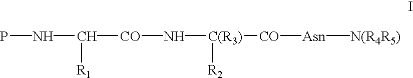

wherein:

P denotes a protecting group or a hydrogen atom,

R₁ denotes

a phenylmethyl radical wherein the phenyl nucleus is sustituted in the para position by a phosphate, phosphonomethyl, phosphonomonofluoromethyl or phosphonodifluoromethyl radical or

a naphtylmethyl radical which may be substituted in the 4 position by a phosphate, phosphonomethyl, phosphonomono- or phosphonodifluoromethyl radical,

each of these radicals also being optionally substituted by one or more substituents selected from among the C₁ to C₄ alkyl or C₁ to C₄ alkoxy groups and/or one or more halogen atoms,

R₂ denotes:

a phenylmethyl or naphtylmethyl or cyclohexylmethyl radical, 2- or 3-pyridinylmethyl, substituted at the para or meta position of the ring by a phosphate, C₁ to C₂ phosphonoalkyl group, preferably phosphonomethyl, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or

a radical alkyl of the type (CH₂)ₙ (wherein n=3 or 4) substituted in end position by a phosphate group, C₁ to C₂ phosphonoalkyl, preferably phosphonomethyl, phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical.

R₃ denotes a straight chain or branched C₁ to C₄ alkyl group or an alkylcycloalkyl group having a C₃ to C₆ cycloalkyl.

R₄ and/or R₅ denote

a hydrogen,

a straight chain or branched C₁ to C₆ alkyl group

a C₁ to C₆ arylalkyl group wherein aryl denotes a phenyl or naphtyl nucleus optionally substituted by one or more hydroxyl groups, or

an aminohexanoic chain followed by the sequences RQIKIWFQNRRMKWKK (SEQ ID N° 1), IRQPKIWFPNRRKPWKK (SEQ ID N° 2), Cys-S-S-Cys-RQIKIWFQNRRMKWKK (SEQ ID N° 3) and Cys-S-S-Cys-IRQPKIWFPNRRKPWKK derived from Antennapedia (SEQ ID N° 4) and the pharmaceutically acceptable salts thereof.

For the purposes of the present invention, the term salts refers to all the addition salts of acids and/or bases which may be obtained from the compounds claimed and which prove to be pharmaceutically acceptable.

They may be alkali metal or alkaline earth metal salts, ammonium salts and organic amine salts. As for acid addition salts they may be derived from hydrohalic acids or other organic acids.

The protecting group P according to the invention may be in particular the (straight chain or branched) aminoalkylcarbonyl, (straight chain or branched), aminoalkoxycarbonyl, (straight chain or branched), arylalkylcarbonyl and (straight chain or branced) arylalkoxycarbonyl groups.

Unexpectedly, the inventors have found that the presence of a ring, preferably an aromatic ring, carrying an acid substituent in the para-position, preferably of a phosphorylated nature in the compounds of general formula I, as R₂, was advantageous in terms of its affinity for the SH2 domain of the protein Grb2. It is found that this effect is reinforced by the presence of a bulky substituent on the α carbon, represented by R₃.

Moreover, the presence of a group derived from phosphonoalkyl groups as a substituent at R₁ and/or R₂ is particularly useful provided that this group is not prone to degradation by phosphatases.

As for the presence of a derivative of Antennapedia derivative as the substituent R₄ and/or R₅, this is advantageous for promoting the cell penetration of said compounds.

According to a preferred embodiment of the invention, the compounds claimed correspond to general formula I wherein:

P denotes an RCO or ROCO group wherein R denotes a C₁₋₄ aminoalkyl or C₁₋₄ aminophenylalkyl,

R₁ denotes a phenylmethyl group substituted in the para position by a substituent selected from among OPO₃H₂, CH₂PO₃H₂, CHFPO₃H₂ and CF₂PO₃H₂,

R₂ denotes a phenylmethyl group substituted in the meta or para position by a substituent as defined hereinbefore in general formula I,

R₃ denotes a C₁ to C₄ alkyl group,

R₄ and/or R₅ denote a hydrogen atom, an alkyl (CH₂)n-CH₃ or (CH₂)ₙ—Ar group wherein Ar denotes a phenyl or α,β-naphthyl which may or may not be substituted and n is between 0 and 5 and the pharmaceutically acceptable salts thereof.

In a preferred embodiment of the invention,

R₁ denotes a phenylmethyl group having a phosphate group in the para-position,

R₂ denotes a phenylmethyl group having, in the para- or meta-position, a group selected from among a phosphate, phosphonomethyl, 2-malonyloxy or (CH₂)ₙCO₂H group wherein n is equal to 0 or 1,

R₃ denotes a C₁-C₄ alkyl group, preferably methyl, and

R₄ and R₅ both represent a hydrogen atom, and the salts thereof.

The following compounds may be mentioned in particular as examples of the pseudopeptide compounds claimed:

mAZ-pTyr-(αMe)pTyr-Asn-NH₂

mAZ-pTyr-(αMe)pTyr-Asn-Aha-Antennapedia

mAZ-Pmp-(αMe)pTyr-Asn-NH₂

mAZ-pTyr-(αMe)Phe(COOH)-Asn-NH₂

mAZ-pTyr-(αMe)Phe(CH₂—COOH)-Asn-NH₂

mAZ-pTyr-(αMe)Pmp-Asn-NH₂

mAZ-pTyr-(αMe)F₂Pmp-Asn-NH₂

mAZ-pTyr-(αMe)Phe(PO₃H₂)-Asn-NH₂

mAZ-pTyr-(αMe)Phe(PO₃H₂)-Asn-Aha-Antennapedia

The present invention also relates to the pseudopeptide compounds of general formula II as prodrugs of the compounds of formula I.

In fact, the phosphate, phosphonate, phosphinate and carboxylate derivatives as claimed hereinafter may be envisaged in the form of prodrugs according to general formula II:

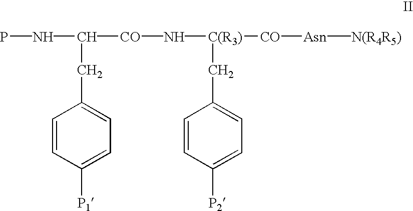

Wherein P, R₃, R₄ and R₅ are as defined in general formula I, and the phenylmethyl groups substituted by P₁′ and P₂′ are the precursors of the groups R₁ and R₂.

More precisely,

the groups P₁′ and/or P₂′ which are precursors of the phosphate group in R₁ and/or R₂ may be either a mono- or bis-(S-acyl-2-thioethyl)phosphate group or a mono- or bis-(acyloxymethyl)phosphate group,

the groups P₁′ and/or P₂′, which are precursors of the phosphonomethyl group in R₁ and/or R₂, may be either a mono- or bis-(S-acyl-2-thioethyl)phosphonomethyl group, or a mono- or bis-(acyloxymethyl)phosphonomethyl group,

the group P₂′, precursor of the phosphonate group in R₂, may be either a mono- or bis-(S-acyl-2-thioethyl)phosphonate group or a mono- or bis-(acyloxymethyl)phosphonate group,

wherein the term acyl in these definitions denotes a tert.butylcarbonyl or isopropylcarbonyl or acetyl group,

the group P₂′, a precursor of the carboxylic acid, carboxymethyl, carboxymethyloxy, malonyl, 2-malonyloxy, 2-(dicarboxy)ethyl groups in R₂, may be an analogue esterified on the carboxylic acid function or functions of the groups R₂ identified above, in the form of a carboxylate of:

arylalkyl where the term aryl denotes a benzene nucleus and the term alkyl denotes a straight or branched carbon chain having 1 to 3 carbon atoms;

morpholinyl alkyl —(CH₂)ₙ(NC₄H₈O);

piperidinyl alkyl —(CH₂)ₙ(NC₅H₁₀) optionally substituted by an OH, CO₂H, CO₂R′ where R′ is a straight or branched alkyl chain which may or may not contain a benzyl or phenyl group;

piperazinylalkyl —(CH₂)ₙ(NC₄H₈NH) optionally substituted by (—N—C₄H₈—NR″) where R″ denotes an alkyl chain containing 1 to 6 carbon atoms, a benzyl group or a phenyl group, wherein n is between 1 and 3.

As for the compounds claimed within the scope of the present invention, these may be prepared by known methods.

Generally, the different pseudopeptide motifs are fused by reacting a reactive group of a first pseudopeptide motif such as, for example, a carboxyl function, with a complementary reactive group such as an amine function carried by a second pseudopeptide motif.

Consequently, the synthesis of the compounds claimed is within the capabilities of the skilled person.

The new compounds have the feature of possessing a peptidomimetic structure which has, at position 1, a phosphotyrosine residue or a substitute carrying one or two negative charges and an optionally positively charged protection for the N-terminal nitrogen and having in position 2, a sterically hindered residue of the alpha-alkyl-phosphotyrosine type or a substitute of the alpha-alkyl phenylalanine or alpha-alkyl cyclohexylalanine type carrying, on the nucleus, in the meta- or para-position, a strongly electron attracting substituent such as an acid function or a methylphosphonate CH₂PO₃H₂, phosphate OPO₃H₂, phosphonate PO₃H₂, sulfonate SO₃H or tetrazolyl c(CN₄H).

As a result of this pseudopeptide organisation, the compounds of general formulae I and II claimed are capable of effectively inhibiting interactions between proteins having a bonding domain SH2, such as Grb2, and phosphorylated proteins such as receptors of growth factors with a tyrosine kinase activity such as the EGF receptor. The compounds of general formulae I and II block the ability of the tyrosine kinase proteins to initiate the signalling cascade via the proteins with an SH2 domain. This results in inhibition of the signal transmission pathways involved in tumoral diseases.

The results given in the examples which follow show the affinity manifested by compounds of formula I for the SH₂ domain of the protein Grb2 and hence their ability to intervene in the reaction thereof with receptors having a tyrosine kinase activity.

Consequently, the compounds of general formula I and general formula II are particularly useful for treating diseases which respond to inhibition of the interaction of proteins having SH2 domains with phosphoproteins, particularly the phosphorylated tyrosine proteins.

The clinical applications for which the compounds according to the invention are particularly intended are the treatment of cancers, metastases and/or diseases connected with proliferative processes.

As examples of the diseases linked with proliferative processes, mention may be made in particular of the pathologies connected with the angiogenesis mechanisms, rheumatoid arthritis, inflammatory diseases and disorders connected with vascular thickening during restenosis.

For these purposes, the compounds claimed and their derivatives may be used for the preparation of corresponding pharmaceutical compositions.

Thus, according to another aspect, the present invention relates more particularly to a pharmaceutical composition containing as active principle at least one compound of general formula (I) or a compound of general formula II.

Of course, this compound may be combined with at least one pharmaceutically acceptable carrier.

Similarly, it is possible to use two or more compounds of general formula I and/or II jointly in a single pharmaceutical composition.

These pharmaceutical compositions may be administered by oral, parenteral or transdermal route.

As for administration by oral route, plain or coated tablets, gelatine capsules, granules, drops or liposome preparations, optionally with delayed release, may be used, in particular.

For administration by intravenous, subcutaneous or intramuscular route, sterile or sterilisable solutions may be used, in particular, for venous perfusion, whereas conventional patches may be produced for administration by transdermal route.

The pharmaceutical compositions according to the present invention may be prepared by conventional methods well known in the field of pharmaceutical technology.

The active principle may be incorporated in the excipients normally used in these pharmaceutical compositions such as talc, gum arabic, lactose, starch, magnesium stearate, aqueous or non-aqueous carriers, animal or vegetable fats, paraffin derivatives, glycols, various wetting, dispersing or emulsifying agents, preservatives, etc.

The quantity of active ingredient to be administered per day will depend of course on the nature of the therapeutic indication, the seriousness of the complaint to be treated as well as the patient's body weight and the route of administration.

As a guide, the compounds of general formulae I and II may be given in a quantity of 10 to 100 mg/kg depending on the route of administration used.

The present invention has also for object a method for the treatment of cancers, metastases and/or diseases connected with proliferative processes comprising administering to a patient in need of such treatment a therapeutically efficient amount of a claimed compound of formula I or II.

The present invention also relates to an automatable process for evaluating the affinity of a compound according to the invention for Grb2, in a rapid throughput test, characterised in that said compound is made to compete with the peptide biotine Aha-PSp-YVNVQN for Grb2 is an ELISA test.

The examples given below are provided as an illustration and should not restrict the present invention.

ABBREVIATIONS

| | |
|---|---|
| Ac₆c: | 1-amino cyclohexyl |
| Aha | 6-aminohexanoic acid |
| Asn: | asparagine |
| Boc: | tert-butyloxycarbonyl |
| DCC: | N,N′-dicyclohexylcarbodiimide |
| DIEA: | diisopropylethylamine |
| DMF: | N,N-dimethylformamide |
| Fmoc: | 9-fluorenylmethoxycarbonyl |
| F₂Pmp: | phosphonodifluoromethylphenylalanine |
| Hepes: | N-(2-hydroxyethyl)piperazine-N′-(2-ethanesulphonic acid) |
| HOBt: | 1-hydroxybenzotriazole |
| HMDS: | hexamethyldisiloxane |
| mAZ: | (meta-amino)benzyloxycarbonyl |
| MDPSE: | (methyl diphenylsilyl)ethyl |
| NMP: | N-methyl pyrrolidone |
| ONp: | O-p-nitrophenyl ester |
| Phe: | phenylalanine |
| Pmp: | phosphonomethylphenylalanine |
| pTyr: | phosphotyrosine |
| TFA: | trifluoroacetic acid |
| TFFH: | tetramethylfluoroformamidinium hexafluorophosphate |
| TIPS: | triisopropylsilane |
| Trt: | trityl |
| Tyr: | tyrosine |

### EXAMPLE 1

### mAZ-pTyr-(α-Me)pTyr-Asn-NH₂

1). Fmoc-L-(α-Me)Tyr-OH

The compound L-(α-Me)Tyr-OH (500 mg, 2.56 mmol) is dissolved in 2 equivalents of 0.5N NaOH (10.4 ml, 5.2 mmol), then a solution of the compound Fmoc-Cl (1.35 g, 5.2 mmol) in acetonitrile (5.2 ml) is added and the whole is stirred for 3 hours at ambient temperature. After evaporation of the organic solvent, a 10% aqueous solution of Na₂CO₃ (25 ml) is added and the suspension is washed with ethyl ether (3×50 ml). The aqueous suspension is then acidified to pH 1 with 6N HCl and extracted with chloroform (4×50 ml). The organic phase is then washed successively with 2N HCl (1×50 ml), H₂O (1×50 ml) and a saturated solution of NaCl (1×50 ml) and then dried over Na₂SO₄. After filtration and evaporation to dryness, the residue is purified by column chromatography on silica (eluant: CH₂Cl₂/MeOH/AcOH 9515/1). 1.09 g of Fmoc-L-(α-Me)Tyr-OH are obtained, in the form of a white powder (yield 100%).

Rf=0.58 (CH₂Cl₂/MeOH/AcOH 95/515)

NMR ¹H (DMSO-d6) (δppm/HMDS): 1.15 (s, 3H, α-Me), 2.75 and 3.05 (dd, 2H, CH₂β), 4.25 (m, 2H, 9′-CH₂ of Fmoc), 4.45 (m, 1H, 9′-H of Fmoc), 6.55 and 6.75 (dd, 4H, 2, 3, 5, 6-H of Tyr), 7.15 (s, 1H, NH), 7.30 (t, 2H, 2′, 7′-H of Fmoc), 7.40 (t, 2H, 3′, 6′-H of Fmoc), 7.70 (d, 2H, 4′, 5′-H of Fmoc), 7.85 (d, 2H, 1′, 8′-H of Fmoc), 9.12 (s, 1H, OH).

2). Fmoc-L-(α-Me)Tyr(PO₃Bzl₂)-OH

The compound Fmoc-L-(α-Me)Tyr-OH (417 mg, 1 mmol) is dissolved in anhydrous THF (3 ml) at ambient temperature and under nitrogen. N-methylmorpholine (0.11 ml, 1 mmol) and tert-butyl dimethylsilane chloride (1M in THF, 1,0 ml, I mmol) are added thereto. Stirring is continued for 15 minutes. Then, 1H-tetrazole (210 mg, 3 mmol) and N, N-diisopropyl dibenzylphosphoramidite (1.0 ml, 3 mmol) are added successively thereto and stirring is continued at ambient temperature for 3 hours. The mixture is cooled to 0° C., meta-chloroperoxybenzoic acid is added (56 to 85%, 690 mg) and the suspension becomes clear. Stirring is continued at 0° C. for 1 hour and then at ambient temperature for 30 minutes. The solution is again cooled to 0° C. and 5% NaHSO₃ (25 ml) is added. The mixture is stirred at 0° C. for 30 minutes then brought to ambient temperature within 30 minutes. After evaporation of the organic solvent, the aqueous residue is extracted with ethyl acetate (3×20 ml). The organic phase is then washed successively with 5% NaHSO₃ (3×25 ml), 1M KHSO₄ (2×25 ml), H₂O (1×25 ml) and a saturated solution of NaCl (1×25 ml), then dried over Na₂SO₄. After filtration and evaporation of the solvent, the residue is taken up in dichloromethane (10 ml), the insoluble solid is filtered and the solution is evaporated to dryness, then purified by column chromatography (eluant: CH₂Cl₂/MeOH 20/1). 572 mg of Fmoc-L-(α-Me)Tyr(PO₃Bzl₂)-OH are obtained, in the form of a white powder (yield: 84%).

Rf=0.40 (CH₂Cl₂/MeOH 9/1).

NMR ¹H (DMSO-d6) (δppm/HMDS): 1.25 (s, 3H, α-Me), 3.02 (m, 2H, CH₂β), 4.15 (m, 2H, 9′-CH₂ of Fmoc), 4.30 (m, 1H, 9′-H of Fmoc), 5.07 (d, 4H, CH₂ of Bzl), 6.85 and 6.95 (dd, 4H, 2, 3, 5, 6-H of Tyr), 7,3 (m, 15H, NH+10H aromatic of Bzl and 2′, 3′, 6′, 7′-H of Fmoc), 7.50 and 7.55 (dd, 2H, 4′, 5′-H of Fmoc), 7.80 (m, 2H, 1′, 8′-H of Fmoc).

3). 3-[N-(tert-Butyloxycarbonyl)amino]benzyl Alcohol

3-aminobenzyl alcohol (12.32 g, 0.1 mol) is dissolved in THF (315 ml) and 1N NaOH (315 ml) and cooled to 0° C. Boc₂O (65.5 g, 0.3 mol) is then added. Stirring is continued overnight and the temperature gradually comes back to ambient temperature. After evaporation of the organic solvent, the residue is acidifed to pH 2 with 1 M KHSO₄, then extracted with CH₂Cl₂ (4×200 ml). The organic phase is then washed successively with 1M KHSO₄ (1×300 ml), H₂O (1×300 ml) and a saturated solution of NaCl (1×500 ml) then dried over Na₂SO₄. After filtration and evaporation to dryness, the residue is recrystallised from petroleum ether (300 ml). The precipitate is filtered and dried. 21.2 g of product are obtained, in the form of a white powder (yield 95%).

Rf=0.42 (CH₂Cl₂/MeOH 95/5)

NMR ¹H (DMSO-d6) (δppm/HMDS): 1.42 (s, 9H, tBu), 4.40 (d, 2H, CH₂O), 5.10 (t, 1H, OH), 6.85 (d, 1H, 6-H), 7.12 (t, 1H, 5-H), 7.25 (d, 1H, 4-H), 7.45 (s, 1H, 2-H), 9.23 (s, 1H, NH).

4). 3-[N-(tert-Butyloxycarbonyl)amino]benzyl-4-nitrophenyl Carbonate (Boc-mAZ-ONp)

The compound 3-[N-(tert-butyloxycarbonyl)amino]benzyl alcohol (1.0 g, 4.5 mmol) is dissolved in pyridine (18 ml) and the solution is cooled to 0° C. Then 4-nitrophenyl chloroformate (0.91 g, 4.5 mmol) is added and stirring is continued overnight during which time the solution comes back to ambient temperature. After evaporation of the solvent in vacuo, the residue is purified by column chromatography (eluant: dichloromethane). 644 mg of product are obtained in the form of a white powder (yield: 37%).

Rf=0.35 (CH₂Cl₂)

NMR ¹H (DMSO-d6) (δppm/HMDS): 1.40 (s, 9H, tBu), 5.23 (s, 2H, CH₂O), 7.00 (d, 1H, 6-H), 7.25 (t, 1H, 5-H), 7.36 (d, 1H, 4-H), 7.59 (s, 1H, 2-H), 7.53 (d, 2H, 2′, 6′-H), 8.25 (d, 2H, 3′, 5′-H), 9.40 (s, 1H, NH).

5). mAZ-pTyr-(α-Me)pTyr-Asn-NH₂

The peptide is synthesised using a 431A Applied Biosystem synthesiser, programmed for Fmoc chemistry.

After the deprotection of the Fmoc group of the amide Rink resin MBHA (200 mg, 0.1 mmol, Novabiochem) with 20% of piperidine in NMP, the first amino acid in the form of Fmoc-Asn(Trt)-OH is coupled with the mixture of DCC/HOBt in the solvent NMP for 1 hour. The Fmoc group is again deprotected with 20% of piperidine in NMP. Because of the hindrance of the α-methyl group, the second amino acid in the form of Fmoc-(α-Me)Tyr(PO₃Bzl₂)-OH (0.5 mmol) is coupled by a more effective coupling agent, TFFH (0.5 mmol), in the presence of DIEA (1.0 mmol) in the solvent DMF (4 ml). A cartridge is prepared containing all these compounds and they are transferred into the reactor containing the peptide resin. Coupling is continued for 4 hours.

Then, the group Fmoc is deprotected and the third amino acid in the form of Fmoc-Tyr(PO₃MDPSE₂)-OH (0.5 mmol) is coupled in the same way using TFFH (0.5 mmol)/DIEA (1.0 mmol) in DMF (4 ml) for 4 hours. After deprotection of the Fmoc group with 20% of piperidine in NMP, the compound Boc-mAZ-ONp (1 mmol) is coupled in NMP in the presence of DIEA (1.2 mmol) for 6 hours, then the peptide resin is washed with NMP and dichloromethane. The peptide resin is dried in vacuo and then suspended in a mixture of TFA/TIPS/H₂O (19 ml/0.5 ml/0.5 ml), stirring for 30 minutes at 0° C., then for 3 hours at ambient temperature.

The resin is then filtered and washed with TFA. The solution is evaporated and the residue is precipitated with cold ethyl ether. The suspension is centrifuged, the supernatant is decanted. The precipitate is then resuspended in cold ether and centrifuged again. The precipitate is then dissolved in H₂O (50 ml) and lyophilised. The crude peptide obtained is then purified over a C18 column (Vydac, 5μ, 10×250 mm) by semi-preparative HPLC (Waters 600 controler) with a UV detector at 220 nm (Waters 480) and the mobile phases A (H₂O+0.1% TFA) and B (70% CH₃CN +0.09% TFA), at a flow rate of 2 ml/min, and a gradient of 100% A over a period of 20 minutes, then increased to 15% B for 100 minutes. The purified fractions are then analysed by HPLC (Shimadzu LC-91) on a C18 analytical column (Vydac, 5μ, 4.6×150 mm) with a UV detector at 220 nm (Shimadzu SPD-10A), at a flow rate of 1 ml/min. The retention time of the peptide is 8.5 min for a gradient from 5% to 65% B over 30 minutes. 17 mg of the peptide are obtained after lyophilisation of the fractions containing the pure peptide. The structure of the peptide is confirmed by mass spectrometry by electrospray (MS 803.3 for 803.6, M−H⁺+Na⁺) and by NMR spectrometry.

¹H NMR (DMSO-d6+TFA) (δppm/HMDS): 1.23 (s, 3H, α-Me), 2.45-2.55 (m, 2H, CH₂β of Asn), 2,70 and 3.00 (mm, 2H, CH₂β of pTyr), 3.05 (m, 2H, CH₂β of (α-Me)pTyr), 4.02 (m, 1H, CHα of Asn), 4.25 (m, 1H, CHα of pTyr), 4.95 (q, 2H, CH₂O of mAZ), 6.9-7.4 (m, 1H, H—Ar), 7.68 (d, 1H, NH of Asn), 7.85 (d, 1H, of pTyr), NH 8.30 (s, 1H, NH of (α-Me)pTyr).

### EXAMPLE 2

### mAZ-pTyr-(α-Me)Tyr-Asn-NH₂ (Control Compound)

This peptide is synthesised in the same way as the peptide mAZ-pTyr-(α-Me)pTyr-Asn-NH₂ with the compound Fmoc-(α-Me)Tyr-OH replacing Fmoc-(α-Me)Tyr(PO₃Bzl₂)-OH.

MS: 723.0 for 700.61, M−H⁺+Na⁺

NMR: ¹H (DMSO-d6+TFA) (δppm/HMDS): 1.22 (s, 3H, α-Me), 2.45-2.55 (m, 2H, CH₂β of Asn), 2,70 and 3.05 (m, 4H, CH₂β of pTyr and of (α-Me)pTyr), 4.00 (m, 1H, CHα of Asn), 4.22 (m, 1H, CHα of pTyr), 4.95 (q, 2H, CH₂O of mAZ), 6.6-7.4 (m, 1H, H—Ar), 7.65 (d, 1H, NH of Asn), 7.85 (d, 1H, NH of pTyr), 8.20 (s, 1H, NH of (α-Me)pTyr).

### EXAMPLE 3

### mAZ-pTyr-pTyr-Asn-NH₂ (Control Compound)

This peptide is synthesised in the same way as the peptide mAZ-pTyr-(α-Me)pTyr-Asn-NH₂, except that the three amino acids are coupled using the mixture DCC/HOBt.

MS: 789.1 for 766.56, M−H⁺+Na⁺

NMR: ¹H (DMSO-d6+TFA) (δppm/HMDS): 2.35-3.0 (m, 6H, CH₂β of Asn and of two pTyr), 4.15 (m, 1H, CHα of Asn), 4.40 (m, 2H, CHα of two pTyr), 4.95 (q, 2H, CH₂O of mAZ), 7.0-7.4 (m, 14H, NH₂ and H—Ar), 7.45 (d, 1H, NH of Asn), 8.20 (t, 2H, NH of two pTyr).

### EXEMPLE 4

### mAZ-pTyr-(αMe)Phe(4-CO₂H)-Asn-NH₂

1). tert-butyl 4-methylbenzoate

A solution of of 4-methyl-benzoyl chloride (10.00 g, 64.7 mmol), in anhydrous ethyl ether (50 ml), is added dropwise to a suspension of lithium tert-butoxide (5.50 g, 68.7 mmol) in tert-butanol (100 ml) at ambient temperature. After one hour's stirring, the solvents are evaporated and the residue is dissolved in ethyl acetate (200 ml). The solution is then washed successively with H₂O (1×50 ml), 1N NaOH (2×50 ml), H₂O (1×50 ml) and a saturated solution of NaCl (1×50 ml), then dried over Na₂SO₄. After filtration and evaporation of the solvent, 12.5 g of product are obtained in the form of a transparent oil (Yield 100%).

Rf=0.64 (AcOEt/c-hexane 1/10).

NMR ¹H (DMSO-d6) (δppm/HMDS): 1.50 (s, 9H, tBu), 2.35 (s, 3H, Me), 7.25 and 7.75 (dd, 4H, Ar—H).

2). tert-butyl (4-bromomethyl)benzoate

A mixture of tert-butyl 4-methylbenzoate (12.00 g, 62.4 mmol), N-bromosuccinimide (12.00 g, 67.4 mmol) and dibenzoyl peroxide (0.82 g, 3.4 mmol) in CCl₄ is refluxed for 3 hours. The solid is filtered and the solvent evaporated. The residue is then taken up in ethyl acetate (200 ml) and washed with H₂O (2×50 ml) and a saturated solution of NaCl (1×50 ml). The organic phase is dried over Na₂SO₄, the solvent is evaporated and the oil obtained is purified by column chromatography on silica (eluant: AcOEt/c-Hexane 1/15). 13.0 g of product 2 are obtained, in the form of a transparent oil (Yield 77%).

Rf=0.42 (AcOEt/c-Hexane 1/15).

NMR ¹H (DMSO-d6) (δppm/HMDS): 1.47 (s, 9H, tBu), 4.65 (s, 2H, CH₂Br), 7.50 and 7.85 (dd, 4H, Ar—H).

3). (3S, 5S, 6R)-4-(Benzyloxycarbonyl)-5, 6-diphenyl-3-methyl-2, 3, 5, 6-tetrahydro-4H-1, 4-oxazin-2-one

Benzyl (2R, 3S)-(−)-6-oxo-2, 3-diphenyl-4-morpholine carboxylate (3.00 g, 7.74 mmol) is dissolved in anhydrous THF (200 ml) at −78° C. under nitrogen. Methyl iodide (4.8 ml, 77.4 mmol) is added dropwise to a 1M solution of NaHMDS in THF (9.3 ml, 9.3 mmol). Stirring is continued for half an hour at −78° C. Then ethyl acetate (300 ml) is added and the organic phase is washed successively with H₂O (2×150ml) and a saturated solution of NaCl (1×200 ml), then dried over Na₂SO₄. After filtration and evaporation, the residue is purified by column chromatography on silica (eluant: AcOEt/c-hexane 1/3). 2.10 g of product 3 are obtained, in the form of a white powder (Yield 68%).

Rf=0.60 (AcOEt/c-hexane 1/2).

NMR ¹H (DMSO-d6) (δppm/HMDS): 1.70 (d, 3H, 3-Me), 4.8-5.3 (m, 4H, 3-H, 5-H and CH₂ of Cbz), 6.25 (d, 1H, 6-H), 6.50 (t, 2H, Ar—H), 6.70 (d, 1H, Ar—H), 7.0-7.4 (m, 12H, Ar—H).

4). (3S, 5S, 6R)4-(Benzyloxycarbonyl)-5, 6-diphenyl-3-methyl-3-[(4′-tert-butoxycarbonyl)benzyl]-2, 3, 5, 6-tetrahydro-4H-1, 4-oxazin-2-one.

Compound 3 (1.00 g, 2.5 mmol) is dissolved in anhydrous THF (20 ml), at a temperature of −78° C. under nitrogen. A 0.5M solution of KHMDS in toluene (15 ml, 97.5 mmol) is added dropwise thereto. After less than 5 minutes, a solution of compound 2 (4.0 g, 14.7 mmol) in anhydrous THF (10 ml) is added dropwise. Stirring is continued for half an hour at −78° C. Then it is worked up using the same method as described for synthesising compound 3. The crude product is then purified by column chromatography on silica (eluant: AcOEt/c-Hexane 1/10). 650 mg of product 4 are obtained, in the form of a white powder (Yield 44%).

Rf=0.32 (AcOEt/c-Hexane 1/10).

NMR ¹H (DMSO-d6) (δppm/HMDS): 1.50 (s, 9H, tBu), 1.80 (s, 3H, 3-Me), 3.25 and 3.95 (dm, 2H, CH₂), 4.55 (s, 1H, 5-H), 5.55 (s, 2H, CH₂ of Cbz), 5.70 (d, 1H, 6-H), 6.70 (m, 4H, Ar—H), 7.0-7.3 (m, 13H, NH and Ar—H), 7.80 (d, 2H, A—-H).

5). (S)-α-Methyl-4-(tert-butoxycarbonyl)phenylalanine

Compound 4 (60 mg, 0.101 mmol) is dissolved in a mixture of THF/EtOH (1/1, 4 ml), 10% palladium on charcoal is added thereto (6 mg) and the suspension is hydrogenated at normal pressure overnight. The catalyst is filtered through celite and the solvent is evaporated off. The residue is precipitated with ether and centrifuged. 28 mg of product 5 are obtained, in the form of a white powder (yield 99%).

NMR ¹H (DMSO-d6+TFA) (δppm/HMDS): 1.40 (s, 3H, α-Me), 1.48 (s, 9H, tBu), 3.05 (q, 2H, CH₂), 7.25 and 7.80 (dd, 4H, Ar—H), 8.25 (s, 3H, NH3⁺).

6). (S)-N-Fluorenylmethoxycarbonyl-α-methyl-4-(tert-butoxycarbonyl) phenylalanine

Compound 5 (188 mg, 0.673 mmol) is dissolved in a mixture of dioxane/NaHCO3 10% (1/1, 30 ml), then Fmoc-Cl (500 mg, 1.93 mmol) is added. The mixture is stirred for 3 hours at ambient temperature, then it is acidified with a 10% citric acid solution to pH 2. It is extracted with CH₂Cl₂ (4×25 ml) and the organic phase is washed with H₂O (1×30 ml) and a saturated solution of NaCl (1×30 ml) and dried over Na₂SO₄. After evaporation of the solvent, the crude product is purified by column chromatography on silica (eluant: CH₂Cl₂/MeOH 95/5). 280 mg of product 6) are obtained, in the form of a white powder (yield 83%).

Rf=0.18 (CH₂Cl₂/MeOH 95/5).

NMR ¹H (DMSO-d6) (δppm/HMDS): 1.15 (s, 3H, α-Me), 1.50 (s, 9H, tBu), 2.95 and 3.25 (dd, 2H, CH₂), 4.20 (t, 1H, 9′-H of Fmoc), 4.25 and 4.40 (mm, 2H, 9′-CH₂ of Fmoc), 7.05 (3H, NH and 2, 6-Ar—H of Phe), 7.28 (t, 2H, 2′, 7′-H of Fmoc), 7.35 (t, 2H, 3′, 6′-H of fmoc), 7.65 (m, 4H, 3, 5-Ar—H of Phe and 4′, 5′-H of Fmoc), 7.85 (d, 2H, 1′, 8′-H of Fmoc).

7). mAZ-pTyr-(αMe)Phe(4-CO₂H)-Asn-NH₂

The peptide is synthesised using the same method as described for the peptide in Example 1 (mAZ-pTyr-(α-Me)pTyr-Asn-NH₂).

MS: 751.1 for 728.62, M−H⁺+Na⁺

NMR: ¹H (DMSO-d6+TFA) (δppm/HMDS): 1.18 (s, 3H, α-Me), 2.30-3.22 (m, 6H, CH₂ of Asn, pTyr and (α-Me)pTyr), 4.20 (m, 2H, CHα of Asn and pTyr), 4.85 (q, 2H, CH₂O of mAZ), 7.0-7.5 (m, 1H, H—Ar), 7.65 (d, 1H, NH of Asn), 7.85 (d, 1H, NH of pTyr), 8.20 (s, 1H, NH of (α-Me)Phe).

### EXAMPLE 5

### mAZ-pTyr-(αMe)Phe(4-CH₂CO₂H)-Asn-NH₂

1). tert-butyl (4-bromomethyl)phenyl Acetate

A mixture of 4-bromomethyl phenylacetic acid (9.7 g, 42.34 mmol) and thionyl chloride (100 ml) is refluxed for 3 hours. Then the excess thionyl chloride is evaporated and the solid residue is dried using a vacuum pump. The product is then dissolved in the minimum of CH₂Cl₂ (4 ml) and this solution is added dropwise to a solution of tert-butanol (140 ml) and CH₂Cl₂ (5 ml) cooled to 0° C. Stirring is continued overnight at 4° C. Then CH₂Cl₂ (100 ml) is added and the organic phase is washed successively with H₂O (1×50 ml), NaHCO₃ 10% (1×50 ml), H₂O (1×50 ml) and a saturated solution of NaCl (1×50 ml). The organic phase is dried over Na₂SO₄, and after evaporation of the solvent and drying with a vacuum pump, 10.9 g of solid product are obtained (yield 91%).

Rf=0.70 (CH₂Cl₂).

NMR ¹H (DMSO-d6) (δppm/HMDS): 1.35 (s, 9H, tBu), 3.50 (s, 2H, CH₂CO₂), 4.65 (s, 2H, CH₂Br), 7.20 and 7.35 (dd, 4H, Ar—H).

2). (3S, 5S, 6R)4-(Benzyloxycarbonyl)-5, 6-diphenyl-3-methyl-3-[((4′-tert-butoxycarbonyl)methyl) benzyl]-2,3,5,6-tetrahydro-4H-1, 4-oxazin-2-one.

Compound 2 is prepared using the same method as described for compound 4 of Example 4 (Yield 31%).

Rf=0.25 (EtOAc/c-Hexane 1/10).

NMR ¹H (DMSO-d6) (δppm/HMDS): 1.30 (s, 9H, tBu), 1.85 (s, 3H, 3-Me), 3.1 and 4.0 (dm, 2H, CH₂β), 3.55 (s, 2H, CH₂CO₂), 4.25 (s, 1H, 5-H), 5.1 (m, 3H, CH₂ of Cbz and 6-H), 6.65-7.30 (m, 20H, NH and Ar—H).

3). (S)-α-Methyl-4-[(tert-butoxycarbonyl)methyl]phenylalanine

Compound 3 is prepared using the same method as described for compound 5 of Example 4. (Yield 90%).

NMR ¹H (DMSO-d6) (δppm/HMDS): 1.30 (s, 3H, α-Me), 1.35 (s, 9H, tBu), 3.0 (q, 2H, CH₂β), 3.50 (s, 2H, CH₂CO₂), 7.15 (m, 6H, NH2 and H—Ar).

4). (S)-N-Fluorenylmethoxycarbonyl-α-methyl-4-[(tert-butoxycarbonyl) Methyl] Phenylalanine

Compound 4 is prepared using the same method as described for compound 6 of Example 4. (Yield 50%).

Rf=0.12 (CH₂Cl₂/MeOH 95/5).

NMR ¹H (DMSO-d6) (δppm/HMDS): 1.20 (s, 3H, α-Me), 1.35 (s, 9H, tBu), 3.0 (q, 2H, CH₂β), 3.40 (s, 2H CH₂CO₂), 4.15 (t, 1H, 9′-H of Fmoc), 4.28 (m, 2H, 9′-CH₂ of Fmoc), 6.95 (q, 4H, Ar—H of Phe), 7.26 (t, 2H, 2′, 7′-H of Fmoc), 7.35 (t, 2H, 3′, 6′-H of fmoc), 7.6 (m, 3H, NH and 4′, 5′-H of Fmoc), 7.85 (d, 2H, 1′, 8′-H of Fmoc).

5). mAZ-pTyr-(αMe)Phe(4-CH₂CO₂H)-Asn-NH₂

The peptide 5 is synthesised using the same method as described for the peptide of Example 1 (mAZ-pTyr-(α-Me)pTyr-Asn-NH₂).

MS: 765.0 for 742.65, M−H⁺+Na⁺

NMR: ¹H (DMSO-d6+TFA) (δppm/HMDS) 1.22 (s, 3H, α-Me), 2.35-3.05 (m, 6H, CH₂β of Asn, pTyr and (α-Me)pTyr), 3.42 (s, 2H, CH₂CO₂H), 4.20 (m, 2H, CHα of Asn and pTyr), 4.85 (q, 2H, CH₂O of mAZ), 7.0-7.4 (m, 12H, H—Ar), 7.65 (d, 1H, NH of Asn), 7.75 (d, 1H, NH of pTyr), 8.35 (s, 1H, NH of (α-Me)Phe).

### EXAMPLE 6

### mAZ-Pmp-(α-Me) pTyr-Asn-NH₂

The peptide is synthesised using the same method as described for the peptide of Example 1 (mAZ-pTyr-(α-Me)pTyr-Asn-NH₂) using Fmoc-Pmp(tBu₂)-OH (Liu, W. -Q. Tetrahedron: Asymmetry, 1995, 6: 647-650) instead of Fmoc-pTyr(PO₃MDPSE₂)-OH.

MS 800.2 for 778.64, M−H⁺+Na⁺

### EXAMPLE 7

### mAZ-pTyr-(D,L)(αMe)Pmp-Asn-NH₂

1). (D,L)-(α-Me)Pmp(tBu₂)-OMe

The compound methyl (N-benzylidene)alaninate (1.0 g, 5.2 mmol) is dissolved in 12 ml of CH₂Cl₂. Then KOH (0.44 g, 7.8 mmol), K₂CO₃ (2.16 g, 15.6 mmol), the catalyst benzyl triethylammonium chloride (0.09 g, 0.4 mmol) and a solution of di-tert-butyl (4-bromomethyl)benzyl phosphonate (2.35 g, 6.2 mmol) in CH₂Cl₂ (4 ml) are added. The mixture is stirred at ambient temperature overnight. The salts are then filtered and the filtrate is evaporated to dryness. 2.77g of methyl (N-benzylidene)[4-(di-tert-butylphosphonomethyl)](α-methyl)-(D,L)-phenylalaninate is obtained, which is hydrolysed without purification, in a 5% citric acid solution (27 ml) and THF (27 ml) at RT for 1 hour. The THF is then evaporated and the aqueous residue is washed with Et₂O then neutralised to pH 8 with a saturated solution of NaHCO₃. The solution is then extracted with AcOEt and the organic phase is washed with H₂O and a saturated solution of NaCl and dried over Na₂SO₄. The residue obtained after evaporation of the solvent is purified by chromatography on silica with CH₂Cl₂/MeOH (95/5) as eluant. 1.2 g of product are obtained in the form of colourless oil (yield: 57%).

Rf=0.48 (CH₂Cl₂/MeOH 95/5).

NMR ¹H (DMSO-d₆+TFA) (δppm/HMDS): 1.25 (s, 18H, 2× tBu), 1.40 (s, 3H, α-Me), 2.95 (d, 2H, CH₂-P), 3.0 (s, 2H, CH₂β), 3.65 (s, 3H, MeO), 7.0 and 7.15 (dd, 4H, H—Ar), 8.45 (s, 3H, NH₃⁺).

2). Fmoc-(D,L)-(α-Me)Pmp(tBu₂)-OH

Compound 1 (1.00 g, 2.5 mmol) is dissolved in 13 ml of dioxane and cooled to 10° C. A solution of 1N NaOH (13 ml, 13 mmol) is added thereto.The mixture is stirred for 15 minutes at 10° C. then for 30 minutes at RT. The mixture is neutralised to pH 9 by bubbling CO₂ through it. Then a solution of Fmoc-Cl (1.3 g, 5.0 mmol) in dioxane (13 ml) is added to the mixture. Stirring is continued at RT for 3 hours and the pH of the mixture is kept at 9 by adding DIEA. The dioxane is evaporated and 5% NaHCO₃ (10 ml) is added to the residue and it is washed with Et₂O. Then the aqueous phase is acidified with 5% citric acid and extracted with AcOEt. The organic phase is washed with H₂O and a saturated solution of NaCl and dried over Na₂SO₄. After evaporation of the solvent, the residue is purified by filtration over a silica block and purified by eluting successively with mixtures of CH₂Cl₂/MeOH (98/2, 95/5, 90/10). 719 mg of product are obtained in the form of a white powder (yield: 47%).

Rf=0.44 (CH₂Cl₂/MeOH 98/2)

NMR ¹H (DMSO-d₆+TFA) (δppm/HMDS): 1.25 (s, 21H, 2× tBu and α-Me), 2.85 (d, 2H, CH₂-P), 3.05 (s, 2H, CH₂β), 4.2 (m, 3H, 9′-CH₂ and 9′-H of Fmoc), 6.95 (m, 4H, H—Ar of Pmp), 7.2-7.85 (m, 9H, H—Ar of Fmoc and NH).

3). mAZ-pTyr-(D,L)(α-Me)Pmp-Asn-NH₂

This peptide is synthesised using the same method as described for the peptide mAZ-pTyr-(α-Me)pTyr-Asn-NH₂.

MS: 801.2 for 778.6, M+Na⁺

NMR ¹H (DMSO-d₆+TFA) (δppm/HMDS): 1.1 and 1.25 (ss, 3H, α-Me), 2.5-3.2 (m, 8H, 3× CH₂β and CH₂-P), 4.2 (m, 1H, CHα), 4.35 (m, 1H, CHα), 4.9-5.05 (m, 2H, CH2 of mAZ), 6.9-7.4 (m, 12H, H—Ar), 7.2 (t, 1H, NH), 7.25 and 8.0 (dd, 1H, NH), 8.35 and 8.55 (ss, 1H, NH).

### EXAMPLE 8

### mAZ-pTyr-(D,L)(α-Me)F₂Pmp-Asn-NH₂

1). (D,L)-(α-Me)F₂Pmp(Et₂)-OMe

This compound is prepared using the method described for the preparation of (D,L)-Pmp(tBu₂)-OMe (yield: 62%).

Rf=0.16 (CH₂Cl₂/MeOH 95/5)

NMR ¹H (DMSO-d₆) (δppm/HMDS) 1.15 (t, 9H, α-Me and 2× CH₃CH₂O), 1.85 (s, 2H, NH₂), 2.82 (q, 2H, CH₂β), 3.55 (s, 3H, MeO), 4.02 (q, 4H, 2× CH₃CH₂O), 7.22 and 7.40 (dd, 4H, H—Ar).

2). Fmoc-(D,L)-(α-Me)F₂Pmp(EtH)-OH

This compound is prepared using the method described for the preparation of Fmoc-(D,L)(α-Me)Pmp(tBu₂)-OH, but we obtained the end product in the form of the phosphonic monoester (yield: 41%).

Rf=0.13 [(CHCl₃:MeOH:H₂O:HOAc)/EtOAc 1(7:3:0.6:0.3)/1]

NMR ¹H (DMSO-d₆) (δppm/HMDS): 1.1 (m, 6H, α-Me and CH₃CH₂O), 2.42 and 3.25 (dd, 2H, CH₂β), 3.95 (q, 2H, CH₃CH₉O), 4.25 (m, 2H, 9-CH₂ of Fmoc), 4.50 (m, 1H, 9-H of Fmoc), 7.0-7.8 (m, 13H, H—Ar and NH).

3). Fmoc-(D,L)-(α-Me)F₂Pmp-OH

Compound 2 (320 mg, 0.57 mmol) is dissolved in 5 ml of acetonitrile and the solution is cooled to −20° C. 0.5 ml of thioanisole and trimethylsilane iodide (5.0 g, 25 mmol) are added thereto. The mixture is then stirred for 30 minutes at 0° C. and for 1 hour at ambient temperature, then evaporated to dryness. The residue is taken up in 16 ml of a solution of TFA/H₂O/CH₃CN (1/1/2) and the mixture is stirred for 1 hour at ambient temperature, then evaporated. The residue is taken up in 10% NaHCO₃ (75 ml) and the solution is washed with ethyl ether. The aqueous phase is then acidified to pH 2 with 6N HCl and extracted with AcOEt. The extract is then dried over Na₂SO₄ and the solvent is evaporated. 260 mg of product are obtained in the form of a white powder (yield: 86%).

Rf=0.08 [(CHCl₃:MeOH:H₂O:HOAc)/AcOEt 1 (7:3:0.6:0.3)/1]

NMR ¹H (DMSO-d6+TFA) (δppm/HMDS): 1.12 (s, 3H, α-Me), 2.88 and 3.22 (dd, 2H, CH₂β), 4.22 (m, 2H, 9-CH₂ of Fmoc), 4.48 (m, 1H, 9-H of Fmoc), 7.0-7.85 (m, 13H, H—Ar and NH).

4). mAZ-pTyr-(D,L)(α-Me)F₂Pmp-Asn-NH₂

This peptide is synthesised using the procedure described for the synthesis of the peptide mAZ-pTyr-(α-Me)pTyr-Asn-NH₂. For the TFFH/DIEA coupling, a mixture of HATU/HOAt/DIEA is used, on account of the use of the unprotected phosphate of (α-Me)F₂Pmp.

MS: 837.1 for 814.5, M+Na⁺

NMR ¹H (DMSO-d6+TFA) (δppm) 1.1 and 1.25 (ss, 3H, α-Me), 2.5-3.35 (m, 6H, 3× CH₂β), 4.25 (m, 2H, 2× CHα), 4.9-5.0 (m, 2H, CH2 of mAZ), 7.0-7.4 (m, 12H, H—Ar), 7.7 and 7.75 (dd, 1H, NH), 7.9 and 8.02 (dd, 1H, NH), 8.39 and 8.6 (ss, 1H, NH).

### EXAMPLE 9

### mAZ-pTyr-(D,L)(α-Me)Phe(4-PO₃H₂)-Asn-NH₂

1). Diethyl (4-methyl)phenylphosphonate

Diethylphosphite (8.3 ml, 64.3 mmol), triethylamine (8.9 ml, 64.3 mmol) and tetrakis(triphenylphosphine)palladium (3.38 g, 2.92 mmol) are dissolved in toluene (100 ml) cooled to 0° C. and under nitrogen. (4-bromo)toluene (10 g, 58.5 mmol) is added thereto and the mixture is refluxed at 90° C. for 3 hours. The mixture is then cooled to RT and ethyl ether (250 ml) is added and then the resulting mixture is filtered. The filtrate is evaporated to dryness and the residue is purified by column chromatography (eluant: AcOEt/c-Hexane 1/1). 10.7 g of product are obtained in the form of a yellow oil (yield 80%).

Rf=0.19 (AcOEt/c-Hexane 2/1) NMR ¹H (DMSO-d₆) (δppm/HMDS): 1.20 (t, 6H, 2× CH₃CH₂O), 2.35 (s, 3H, CH₃-Ar), 3.95 (q, 4H, 2× CH₃CH₂O), 7.3-7.6 (m, 4H, H—Ar).

2). Diethyl (4-bromomethyl)phenylphosphonate

Compound 1 (5.0 g, 21.9 mmol), NBS (3.9 g, 21.9 mmol) and dibenzoyl peroxide (266 mg, 1.1 mmol) are dissolved in 50 ml of CCl₄. The mixture is refluxed for 3 hours, then cooled to RT. The precipitate is filtered and the filtrate is evaporated to dryness. The residue obtained is dissolved in ethyl acetate and the solution is washed with H₂O and a saturated solution of NaCl, then dried over Na₂SO₄. After evaporation of the solvent, the residue is purified by column chromatography (eluants: AcOEt/c-Hexane 1/4, 1/2 and 2/1) (yield 88%).

Rf=0.38 (AcOEt/c-Hexane 4/1)

NMR ¹H (DMSO-d₆) (δppm/HMDS) 1.20 (t, 6H, 2× CH₃CH₂O), 4.0 (m, 4H, 2× CH₃CH₂O), 4.70 (s, 2H, CH₂Br), 7.1 (m, 4H, H—Ar).

3). (D,L)-(α-Me)Phe(4-PO₃Et₂)-OMe

This compound is prepared using the method described for the preparation of (D,L)-Pmp(tBu₂)-OMe (yield: 66%).

Rf=0.36 (CH₂Cl₂/MeOH 95/5)

NMR ¹H (DMSO-d₆) (δppm/HMDS): 1.15 (t, 9H, α-Me and 2× CH₃CH₂O), 1.80 (s, 2H, NH₂), 2.85 (q, 2H, CH₂β), 3.55 (s, 3H, MeO), 3.95 (q, 4H, 2× CH₃CH₂β), 7.25-7.55 (m, 4H, H—Ar).

4). Fmoc-(D,L)-(α-Me)Phe(4-PO₃Et₂)-OH

This compound is prepared using the method described for the preparation of Fmoc-(D,L)(α-Me)Pmp(tBu₂)-OH (yield: 23%).

Rf=0.1 (CH₂Cl₂/MeOH 95/5)

NMR ¹H (DMSO-d₆) (δppm/HMDS): 1.20 (t, 6H, CH₃CH₂O), 1.35 (s, 3H, α-Me), 3.15 and 3.30 (dd, 2H, CH₂β), 3.95 (m, 2H, CH₃CH₂O), 4.2 (m, 2H, 9-CH₂ of Fmoc), 4.45 (m, 1H, 9-H of Fmoc), 6.65 (s, 1H, NH), 7.1-7.8 (m, 12H, H—Ar).

5). Fmoc-(D,L)-(α-Me)Phe(4-PO₃H₂)-OH

This compound is prepared by hydrolysing compound 5 using TMSI, by the method described for the preparation of Fmoc-(D,L)-(α-Me)F₂Pmp-OH (yield: 80%).

Rf=0.44 (CHCl₃/MeOH/H₂O/HOAc 71310.610.3)

NMR ¹H (DMSO-d₆+TFA) (δppm) 1.15 (s, 3H, α-Me), 2.9 and 3.25 (dd, 2H, CH₂β), 4.25 (m, 2H, 9-CH₂ of Fmoc), 4.4 (m, 1H, 9-H of Fmoc), 7.0-7.85 (m, 13H, H—Ar and NH).

6). mAZ-pTyr-(L or D)(α-Me)Phe(4-PO₃H₂)-Asn-NH₂

6′). mAZ-pTyr-(D or L)(α-Me)Phe(4-PO₃H₂)-Asn-NH₂

This peptide is synthesised using the procedure described for the synthesis of the peptide mAZ-pTyr-(α-Me)pTyr-Asn-NH₂. When using the TFFH/DIEA coupling method, a mixture of HATU/HOAt/DIEA is used on account of the use of the unprotected phosphate of (α-Me)Phe(4-PO₃H₂). We were able to separate the two diastereoisomers by semi-preparative HPLC.

Peptide 6: Rt=11.0 min (5 to 35% of solvent B over 30 min, C₁₈ column)

NMR ¹H (DMSO-d₆+TFA) (δppm/HMDS): 1.25 (s, 3H, α-Me), 2.5-3.2 (m, 6H, 3× CH₂β), 4.18 (m, 1H, CHα), 4.28 (m, 1H, CHα), 4.95 (q, 2H, CH₂ of mAZ), 7.05-7.5 (m, 12H, H—Ar), 7.7 (d, 1H, NH), 7.9 (d, 1H, NH), 8.3 (s, 1H, NH).

Peptide 6′: Rt=10.0 min (5 to 35% of solvent B over 30 min, C₁₈ column)

NMR ¹H (DMSO-d₆+TFA) (δppm/HMDS): 1.10 (s, 3H, α-Me), 2.6-3.35 (m, 6H, 3× CH₂β), 4.25 (m, 1H, CHα), 4.35 (m, 1H, CHα), 5.0 (q, 2H, CH₂ of mAZ), 7.05-7.5 (m, 12H, H—Ar), 7.7 (d, 1H, NH), 8.1 (d, 1H, NH), 8.55 (s, 1H, NH).

### EXAMPLE 10

### mAZ-pTyr-(α-Me)pTyr-Asn-Aha-IRQPKIWIPNRRKPWKK

This peptide is synthesised using a 431A Applied Biosystem synthesiser, programmed for Fmoc chemistry.

The first amino acid in the form of Fmoc-Lys(Boc)-OH is coupled to the resin HMP (100 mg, 0.089 mmol, Applied Biosystem) with a mixture of DCC/DMAP. The next 18 amino acids are then coupled by repeating the deprotection/coupling steps. The residues (α-Me)pTyr, pTyr and mAZ are introduced by the method described for the synthesis of Example 1 (mAZ-pTyr-(α-Me)pTyr-Asn-NH2). The peptide resin is cleaved with a mixture of TFA/TIPS/PhOH/H2O (20 ml/1 ml/1.5 g/1 ml). The crude peptide precipitated by ether is purified by HPLC on a C18 column (Vydac, 5μ, 10×250 mm), with the mobile phases A (H₂O+0.1% TFA) and B (70% CH₃CN+0.09% TFA), at a flow rate of 2 ml/min, and a gradient to 5% B over 20 minutes, then increased to 35% B for 100 minutes. The purified fractions are then analysed by HPLC on a C18 analytical column (Vydac, 5μ, 4.6×150 mm) at a flow rate of 1 ml/min. The retention time for the peptide is 14.7 min for a gradient of 25% to 35% B over 30 minutes. The structure of the peptide is confirmed by mass spectrometry by electrospray (MS 3041.1 for 3042.3).

### EXAMPLE 11

### Affinities of the Peptidomimetics for Grb2

The affinities of the compounds for Grb2 are measured at 25° C. in a 50 mM HEPES buffer and 1 mM DTT, pH 7.5, by fluorescence, by measuring the emission spectrum at 345 nm (aperture 5.0 nm) under excitation at 292 nm (aperture 5.0 nm), using the procedure described by Cussac and al. (EMBO J., 1994, 13, 4011-4021) and are shown in Table 1. They are expressed therein as the dissociation constant Kd.

Each of the compounds was also tested, using an ELISA test, for its ability to compete with the interaction between Grb2 and a phosphotyrosine peptide from the protein Shc and corresponding to the tyrosine 317 (PSPYVNVQN) the affinity of which for Grb2 was evaluated by fluorescence (Kd=18 nM).

This ELISA test was perfected in order to avoid declaring a compound inactive if it did not induce any variation in fluorescence during the interaction.

Plates pre-treated with streptavidine (Boehringer) are incubated overnight at 4° C. with 100 μl of a solution of peptide biotin-Aha-PSpYVNVQN (Aha: 6-amino-hexanoic acid) (100 nM solution in TBS buffer Tris 100 mM, NaCl 50 mM, pH 7.5) per well. The non-specific binding is then blocked by incubating for 4 hours with the same buffer containing 3% of skimmed milk (800 μl per well). The products to be tested are distributed among the wells at the desired dilution in the abovementioned buffer containing 3% of skimmed milk and 40 ng/ml of GST-Grb2 (100 μl per well). After one night's incubation, the plates are carefully rinsed 4 times using TBS-milk-0.05% tween 20, then incubated for 2 hours at 37° C., in the presence of 100 μl per well of anti-GST antibody (transduction, dilution 1/500 in TBS-milk-0.05% tween 20). The plates are then carefully rinsed 4 times using TBS-milk-0.05% tween 20, then incubated for 45 minutes at 37° C. in the presence of 100 pi per well of anti-mouse antibody coupled with peroxidase (Amersham, 1/1000 dilution in TBS-milk-0.05% tween 20). The plates are carefully rinsed using TBS-milk-0.05% tween 20, then incubated in the presence of 200 μl per well of TMB developing solution (Interchim), until a sufficient blue coloration has developed. The reaction is then stopped by adding 100 μl of 10% (VN) sulphuric acid per well. The reading is taken at 550 nm. The value read off for each well is then reduced by the control value of an equivalent well with no fixed peptide biotin-Aha-PSpYVNVQN then treated, like its homologue, with fixed peptide. The data is then processed using “Origin 40” software to obtain the 50 inhibitory concentrations.

TABLE 1
Affinities measured by fluorescence and IC₅₀ by
ELISA (Grb2/biotin-Aha-PSpYVNVQN displacement).

| | Diss. const. | IC₅₀ |
|---|---|---|
| Product | fluo K_{d} (nM) | (nM) ELISA |
| PSpYVNVQN (Shc 317) | 18 (+/−2) | 71 (+/−5) |
| mAZ-pTyr-Acac-Asn-NH₂ | 30 (+/−5) | 120 (+/−8) |
| mAZ-pTyr-pTyr-Asn-NH₂ | 60 (+/−10) | 235 (+/−42) |
| mAZ-pTyr-Tyr-Asn-NH₂ | 155 (+/−30) | 497 (+/−50) |
| mAZ-pTyr-(αMe)pTyr-Asn-NH₂ *A | 3 (+/−1) | 11 (+/−1) |
| mAZ-pTyr-(αMe)Tyr-Asn-NH₂ | 250 (+/−50) | 1098 (+/−160) |
| mAZ-Tyr-(αMe)pTyr-Asn-NH₂ | rate of | 698 (+/−335) |
| | variation | |
| mAZ-Pmp-(αMe)pTyr-Asn-NH₂ | | 42 (+/−22) |
| mAZ-pTyr-(αMe)Phe(COOH)-Asn-NH₂ | 45 (+/−10) | 153 (+/−38) |
| mAZ-pTyr-(αMe)Phe(CH₂—COOH)- | 60 (+/−10) | 198 (+/−41) |
| Asn-NH₂ | | |
| mAZ-pTyr-(αMe)Pmp-Asn-NH₂ | 70 (+/−30) | 266 |
| mAZ-pTyr-(αMe)F₂Pmp-Asn-NH₂ | | 64 |
| mAZ-pTyr-(αMe)Ppp-Asn-NH₂ (D or L) | 4.5 (+/−4) | 14 |
| mAZ-pTyr-(αMe)Ppp-Asn-NH₂ (D or L) | | 113 |

Pmp: para-phosphonomethyl-phenylalanine
Ppp: para-phosphonophenylalanine
F₂Pmp: para-phosphonodifluoromethyl-phenylalanine

1. A pseudopeptide corresponding to general formula I 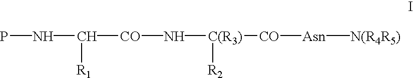
wherein:
P denotes a protecting group or a hydrogen atom,
R₁ denotes
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical,
each of these radicals also being optionally substituted by one or more substituents selected from among the C₁ to C₄ alkyl or C₁ to C₄ alkoxy groups and/or one or more halogen atoms,
R₂ denotes:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical,
R₃ denotes a straight chain or branched C₁ to C₄ alkyl group or an alkylcycloalkyl group having a C₃ to C₆ cycloalkyl,
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or
an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK derived from Antennapedia and pharmaceutically acceptable salts thereof.

2. The compound according to claim 1, wherein:
P denotes an RCO or ROCO group where R denotes a C₁₋₄ aminoalkyl or C₁₋₄ aminophenylalkyl,
R₁ denotes a phenylmethyl group substituted in the para position by a substituent selected from among OPO₃H₂, CH₂PO₃H₂, CHFPO₃H₂ and CF₂PO₃H₂,
R₂ denotes a phenylmethyl group substituted in the meta or para position by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical,
R₃ denotes a C₁ to C₄ alkyl group,
R₄ and/or R₅ denote a hydrogen atom, an alkyl (CH₂)ₙ CH₃ or (CH₂)ₙ—Ar group wherein Ar denotes a phenyl or α,β-naphthyl which may or may not be substituted and n is between 0 and 5 and pharmaceutically acceptable salts thereof.

3. A compound according to claim 1, wherein:
R₁ denotes a phenylmethyl group having a phosphate group in the para-position,
R₂ denotes a phenylmethyl group having, in the para- or meta-position, a group selected from the group consisting of a phosphate, phosphonomethyl, 2-malonyloxy or (CH₂)ₙCO₂H group wherein n is equal to 0 or 1,
R₃ denotes a C₁-C₄ alkyl group, and
R₄ and R₅ both represent a hydrogen atom and the pharmaceutically acceptable salts thereof.

4. The compound according to claim 1 selected from the group consisting of:
mAZ-pTyr-(αMe)pTyr-Asn-NH₂
mAZ-pTyr-(αMe)pTyr-Asn-Aha-Antennapedia
mAZ-Pmp-(αMe)pTyr-Asn-NH₂
mAZ-pTyr-(αMe)Phe(COOH)-Asn-NH₂
mAZ-pTyr-(αMe)Phe(CH₂—COOH)-Asn-NH₂
mAZ-pTyr-(αMe)Pmp-Asn-NH₂
mAZ-pTyr-(αMe)F₂Pmp-Asn-NH₂
mAZ-pTyr-(αMe)Phe(PO₃H₂)-Asn-NH₂
mAZ-pTyr-(αMe)Phe(PO3H2)-Asn-Aha-Antennapedia

5. Pseudopeptide compound corresponding to general formula II: 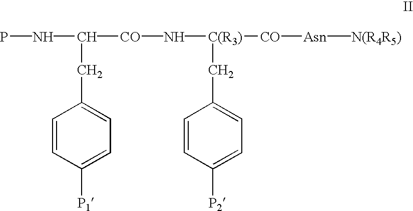
wherein:
P denotes a protecting group or a hydrogen atom,
R₃ denotes a straight chain or branched C₁ to C₄ alkyl group or an alkylcycloalkyl group having a C₃ to C₆ cycloalkyl,
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or
an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK, derived from Antennapedia, and the phenylmethyl group substituted by P₁′ is a precursor of a group selected from the group consisting of:
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical,
each of these radicals also being optionally substituted by one or more substituents selected from the group consisting of C₁ to C₄ alkyl or C₁ to C₄ alkoxy groups and/or one or more halogen atoms,
and the phenylmethyl group substituted by P₂′ is a precursor of a group selected from the group consisting of:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical and the pharmaceutically acceptable salts thereof.

6. The compound according to claim 5, wherein:
P denotes an RCO or ROCO group where R denotes a C₁₋₄ aminoalkyl or C₁₋₄ aminophenylalkyl,
R₃ denotes a C₁ to C₄ alkyl group,
R₄ and/or R₅ denote a hydrogen atom, an alkyl (CH₂)ₙ—CH₃ or (CH₂)ₙ—Ar group wherein Ar denotes a phenyl or α,β-naphthyl which may or may not be substituted and n is between 0 and 5,
the phenylmethyl group substituted by P₁′ is a precursor of a phenylmethyl group substituted in the para position by a substituent selected from the group consisting of OPO₃H₂, CH₂PO₃H₂, CHFPO₃H₂ and CF₂PO₃H₂, and
the phenylmethyl group substituted by P₂′ is a precursor of a phenylmethyl group substituted in the meta or para position by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical.

7. The compound according to claim 5, wherein:
R₃ denotes a C₁-C₄ alkyl group;
R₄ and R₅ both represent a hydrogen atom;
the phenylmethyl group substituted by P₁′ is a precursor of a phenylmethyl group having a phosphate group in the para-position,
the phenylmethyl group substituted by P₂′ is a precursor of a phenylmethyl group having, in the para- or meta-position, a group selected from the group consisting of a phosphate, phosphonomethyl-2-malonyloxy or (CH₂)ₙCO₂H group wherein n is equal to 0 or 1.

8. The compound according to claim 5, wherein the groups P₁′ and/or P₂′ are mono or bis-(S-acyl-2-thioethyl)phosphate and/or mono or bis-(acyloxymethyl) phosphate groups wherein the term acyl denotes a tert.butylcarbonyl or isopropylcarbonyl or acetyl group.

9. The compound according to claim 5, wherein the groups P₁′ and/or P₂′ are mono or bis-(S-acyl-2-thioethyl)phosphonomethyl and/or mono or bis-(acyloxymethyl) phosphonomethyl groups wherein the term acyl denotes a tert.butylcarbonyl or isopropylcarbonyl or acetyl group.

10. Compound according to claim 5, wherein the group P₂′ is a mono or bis-(S-acyl-2-thioethyl)phosphonate and/or mono or bis-(acyloxymethyl)phosphonate group wherein the term acyl denotes a tert.butylcarbonyl or isopropylcarbonyl or acetyl group.

11. Compound according to claim 5, wherein the group P₂′, is in the form of a carboxylate of:
arylalkyl where the term aryl denotes a benzene nucleus and the term alkyl denotes a straight or branched carbon chain having 1 to 3 carbon atoms;
morpholinyl alkyl —(CH₂)ₙ(NC₄H₈O);
piperidinyl alkyl —(CH₂)ₙ(NC₅H₁₀) optionally substituted by an OH, CO₂H, CO₂R′ where R′ is a straight or branched alkyl chain which may or may not contain a benzyl or phenyl group; or
piperazinylalkyl —(CH₂)ₙ(NC₄H₈NH) optionally substituted by (—N—C₄H₈—NR″) where R″ denotes an alkyl chain containing 1 to 6 carbon atoms, a benzyl group or a phenyl group, wherein n is between 1 and 3.

12. A pharmaceutical composition containing as active ingredient at least one compound of general formula I according to claim 1.

13. A pharmaceutical composition containing as active ingredient at least one compound selected from the group consisting of:
mAZ-pTyr-(αMe)pTyr-Asn-N H₂
mAZ-pTyr-(αMe)pTyr-Asn-Aha-Antennapedia
mAZ-Pmp-(αMe)pTyr-Asn-NH₂
mAZ-pTyr-(αMe)Phe(COOH)-Asn-NH₂
mAZ-pTyr-(αMe)Phe(CH₂—COOH)-Asn-NH₂.
mAZ-pTyr-(αMe)Pmp-Asn-N H₂
mAZ-pTyr-(αMe)F₂Pmp-Asn-N H₂
mAZ-pTyr-(αMe)Phe(PO₃H₂)-Asn-NH₂
mAZ-pTyr-(αMe)Phe(PO3H2)-Asn-Aha-Antennapedia

14. A pharmaceutical composition containing as active ingredient at least one compound of general formula II according to claim 5.

15. A method of preparing a pharmaceutical composition intended for the treatment of diseases connected with proliferative processes, cancers and/or metastases comprising combining excipients with a compound of general formula I 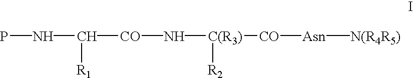
wherein:
P denotes a protecting group or a hydrogen atom,
R₁ denotes
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical,
each of these radicals also being optionally substituted by one or more substituents selected from among the C₁ to C₄ alkyl or C₁ to C₄ alkoxy groups and/or one or more halogen atoms,
R₂ denotes:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical,
R₃ denotes a straight chain or branched C₁ to C₄ alkyl group or an alkylcycloalkyl group having a C₃ to C₆ cycloalkyl,
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or
an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK derived from Antennapedia and pharmaceutically acceptable salts thereof.

16. A method of preparing a pharmaceutical composition intended for the treatment of diseases connected with proliferative processes, cancers and/or metastases comprising combining excipients with a compound selected from the group consisting of:
mAZ-pTyr-(αMe)pTyr-Asn-NH₂
mAZ-pTyr-(αMe)pTyr-Asn-Aha-Antennapedia
mAZ-Pmp-(αMe)pTyr-Asn-NH₂
mAZ-pTyr-(αMe)Phe(COOH)-Asn-NH₂
mAZ-pTyr-(αMe)Phe(CH₂—COOH)-Asn-NH₂.
mAZ-pTyr-(αMe)Pmp-Asn-NH₂
mAZ-pTyr-(αMe)F₂Pmp-Asn-NH₂
mAZ-pTyr-(αMe)Phe(PO₃H₂)-Asn-NH₂
mAZ-pTyr-(αMe)Phe(PO3H2)-Asn-Aha-Antennapedia.

17. A method of preparing a pharmaceutical composition intended for the treatment of diseases connected with proliferative processes, cancers and/or metastases comprising combining excipients with a compound of general formula II 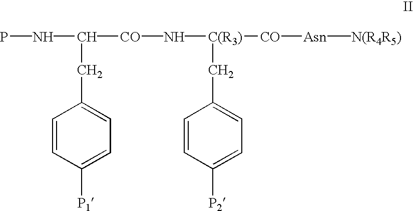
wherein:
P denotes a protecting group or a hydrogen atom,
R₃ denotes a straight chain or branched C₁ to C₄ alkyl group or an alkylcycloalkyl group having a C₃ to C₆ cycloalkyl.
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or
an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK, derived from Antennapedia,
and the phenylmethyl group substituted by P₁′ is a precursor of a group selected from the group consisting of:
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical,
each of these radicals also being optionally substituted by one or more substituents selected from the group consisting of C₁ to C₄ alkyl or C₁ to C₄ alkoxy groups and/or one or more halogen atoms,
and the phenylmethyl group substituted by P₂′ is a precursor of a group selected from the group consisting of:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical and the pharmaceutically acceptable salts thereof.

18. A method for the treatment of cancers, metastases and/or diseases connected with proliferative processes comprising administering to a patient in need of such treatment a therapeutically efficient amount of a compound comprising a pseudopeptide corresponding to general formula I 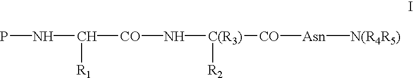
wherein:
P denotes a protecting group or a hydrogen atom,
R₁ denotes
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical, each of these radicals also being optionally substituted by one or more substituents selected from among the C₁ to C₄ alkyl or C₁ to C₄ alkoxy groups and/or one or more halogen atoms,
R₂ denotes:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical,
R₃ denotes a straight chain or branched C₁ to C₄ alkyl group or an alkylcycloalkyl group having a C₃ to C₆ cycloalkyl.
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK derived from Antennapedia and pharmaceutically acceptable salts thereof.

19. A method for the treatment of cancers, metastases and/or diseases connected with proliferative processes comprising administering to a patient in need of such treatment a therapeutically efficient amount of a compound from the group consisting of:
m-AZ-pTyr-(αMe)pTyr-Asn-NH₂
mAZ-pTyr-(αMe)pTyr-Asn-Aha-Antennapedia
mAZ-Pmp-(αMe)pTyr-Asn-NH₂
mAZ-pTyr-(αMe)Phe(COOH)-Asn-NH₂
mAZ-pTyr-(αMe)Phe(CH₂—COOH)-Asn-NH₂.
mAZ-pTyr-(αMe)Pmp-Asn-NH₂
mAZ-pTyr-(αMe)F₂Pmp-Asn-NH₂
mAZ-pTyr-(αMe)Phe(PO₃H₂)-Asn-NH₂
mAZ-pTyr-(αMe)Phe(PO3H2)-Asn-Aha-Antennapedia

20. A method for the treatment of cancers, metastases and/or diseases connected with proliferative processes comprising administering to a patient in need of such treatment a therapeutically efficient amount of a compound comprising a pseudopeptide compound corresponding to general formula II: 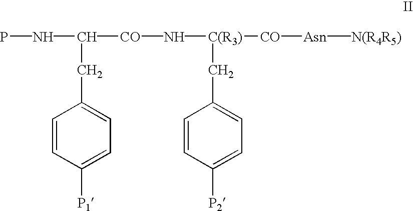
wherein:
P denotes a protecting group or a hydrogen atom,
R₃ denotes a straight chain or branched C₁ to C₄ alkyl group or an alkylcycloalkyl group having a C₃ to C₆ cycloalkyl.
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or
an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK, derived from Antennapedia,
and the phenylmethyl group substituted by P₁′ is a precursor of a group selected from the group consisting of:
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical,
each of these radicals also being optionally substituted by one or more substituents selected from the group consisting of C₁ to C₄ alkyl or C₁ to C₄ alkoxy groups and/or one or more halogen atoms,
and the phenylmethyl group substituted by P₂′ is a precursor of a group selected from the group consisting of:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical and the pharmaceutically acceptable salts thereof.

21. An automatable process for evaluating, in a high throughput test, the affinity of a compound comprising a pseudopeptide corresponding to general formula I 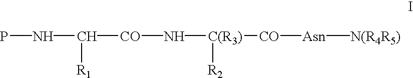
wherein:
P denotes a protecting group or a hydrogen atom,
R₁ denotes
a phenylmethyl radical wherein the phenyl nucleus is substituted in the para position by a phosphate, phosphonomethyl, phosphonomonofluoromethyl or phosphonodifluoromethyl radical or
a naphtylmethyl radical which may be substituted in the 4 position by a phosphate, phosphonomethyl, phosphonomono- or phosphonodifluoromethyl radical, each of these radicals also being optionally substituted by one or more substituents selected from among the C₁ to C₄ alkyl or C₁ to C₄ alkoxy groups and/or one or more halogen atoms,
R₂ denotes:
a phenylmethyl or naphtylmethyl or cyclohexylmethyl radical, 2- or 3-pyridinylmethyl, substituted at the para or meta position of the ring by a phosphate, C₁ to C₂ phosphonoalkyl group, phosphonomonofluoromethyl, phosphonodifluoromethyl, phosphonate, phosphinate, sulfonate, sulfonomethyl, carboxylate, carboxymethyl, carboxymethyloxy, malonyl, 2-(dicarboxy)ethyl, 2-malonyloxy, 5-tetrazolyl or 5-tetrazolylmethyl radical or
a radical alkyl of the type (CH₂)ₙ (wherein n=3 or 4) substituted in end position by a phosphate group, C₁ to C₂ phosphonoalkyl, preferably phosphonomonofluoromethyl and phosphonodifluoromethyl, phosphonate, phosphinate, sulfonate, sulfonomethyl, carboxylate, carboxymethyl, carboxymethyloxy, malonyl, 2-malonyloxy, 2-dicarboxyethyl, 5-tetrazolyl or 5-tetrazolylmethyl radical,
R₃ denotes a straight chain or branched C₁ to C₄ alkyl group or an alkylcycloalkyl group having a C₃ to C₆ cycloalkyl,
R₄ and/or R₅ denote
a hydrogen,
a straight chain or branched C₁ to C₆ alkyl group
a C₁ to C₆ arylalkyl group wherein aryl denotes a phenyl or naphtyl nucleus optionally substituted by one or more hydroxyl groups, or an aminohexanoic chain followed by the sequences RQIKIWFQNRRMKWKK, IRQPKIWFPNRRKPWKK, Cys-S-S-Cys-RQIKIWFQNRRMKWKK and Cys-S-S-Cys-IRQPKIWFPNRRKPWKK derived from Antennapedia and pharmaceutically acceptable salts thereof for Grb2, wherein said compound is made to compete with the peptide biotine Aha-PSpYVNVQN for Grb2 in an ELISA test.
